(19) United States
(12) Patent Application Publication
(10) Pub. No.: US 2024/0081679 A1
(43) Pub. Date: Mar. 14, 2024

(54) METHOD FOR DETERMINING THE LOCATION AND POSITION OF A PERSON'S PELVIS

(71) Applicant: SANLAS HOLDING GMBH, Graz (AT)

(72) Inventor: Cornelia ZENZMAIER, Laßnitzhöhe (AT)

(21) Appl. No.: 17/754,752

(22) PCT Filed: Sep. 24, 2020
(86) PCT No.: PCT/AT2020/060343

§ 371 Date: Apr. 11, 2022

(30) Foreign Application Priority Data
Oct. 16, 2019 (AT) A 50895/2019

(51) Int. Cl. A61B 5/103; A61B 5/00

(52) U.S. Cl. CPC A61B 5/1036; A61B 5/6891; A61B 5/6892; A61B 2562/0247

(57) ABSTRACT

A method of determining the place and location of a human pelvis, including detecting sitting pressures/contact pressures exerted on the seat, the lounger or the support by the ischial tuberosities and the coccyx of the sitting person or by the iliac crests and the pubic bone of the lying person by a surface sensor; determining, by a computer unit, a first position on the surface sensor where a first peak sitting pressure/peak contact pressure is reached; determining a second position on the surface sensor where a second peak sitting pressure/peak contact pressure is reached; determining a distance between the first position and the second position; determining a third position on the surface sensor where a third peak sitting pressure/peak contact pressure is reached, the position being between the first position and the second position; and limiting an area of the surface sensor which detects sitting and contact pressures.

The present invention relates to a method and a device for determining the place and location of a pelvis of a person sitting on a seat or sitting or lying on a lounger or a support.

A person's pelvis is considered to be the position controller for a neutral orientation of the functional spine and the lower extremities. Although the pelvis shows age- and gender-specific differences in terms of length and angular ratios, a universally valid, uniform description can still be given for its location-related stress. In seating systems, the person's pelvis is generally supported by the seating surface and the backrest of the seating system. The ischial tuberosities thereby serve for absorbing the weight of the upper body and portions of the lower extremities and can absorb a high sitting pressure when the person is in an ergonomically correct, essentially upright sitting position, whereas the coccyx and the sacrum remain essentially unstressed in this upright sitting position. With reclining systems in an ergonomically correct horizontal dorsal position of the person, the spine and the sacrum support the weight force of the upper body, whereby the contact pressure exerted by the weight force of the person in the pelvic area is substantially at a maximum on the sacrum and the coccyx remains essentially unstressed. In an ergonomically correct prone position, the person's pubic bone takes on the maximum portion of that body section weight in the pelvic area.

However, people are often not aware of their own individual posture when standing, sitting and lying down and adopt positions that can cause one-sided painful muscle contractures and, in the long run, can lead to attrition processes, including herniated discs, or at least to compressions and sprains, especially of the spine, in the event of incorrect loading of the spine and pelvis structure, especially in the transition areas thereof, the sacroiliac joints. Sitting for particularly long periods of time requires permanent static work from the muscles, often in a false posture and without sufficient compensatory movements, which causes muscular imbalances and promotes a wide range of physical complaints, also in the extremities.

Numerous means and methods are known from the prior art which strive to create and continually maintain an ergonomically correct posture. For instance, DE 10 2012 017 681 A1, US 2002/0193707 A1 and EP 1 093 755 A1, for example, show methods and devices for measuring contact pressures of a person when sitting or lying down, respectively. However, with such methods and, respectively, devices, it is not possible to perform a determination of the location and place of a person's pelvis.

For example, patent application A 50386/2019, which has not yet been published at the time of the present patent application, discloses a device for positioning a person's body with control elements and sensors arranged on a seat element and on a back element. In this patent application, the rolling process of the person during his or her transfer from an upright to a horizontal reclining position is mentioned, with the person's pelvis being positioned by linear adjusting movements via rotations about the sagittal, horizontal and longitudinal axes.

In patent application A 50386/2019, the location of the pelvis is determined only indirectly and with insufficient accuracy, since the person's pelvis is already positioned as soon as a predetermined sifting and/or contact pressure difference is reached. Moreover, this device fails to detect mixed movements, i.e., movements occurring simultaneously around at least two body axes of the person, but merely detects a movement exclusively around the sagittal, horizontal or longitudinal axis. However, in order to achieve ergonomic positioning of the person on the seating or reclining system, it is necessary to accurately determine the location of the pelvis in every position of the person on the seating or reclining system.

It is therefore the object of the present invention to provide a method and a device for determining the location and place of a person's pelvis, which can be integrated into a pre-existing seating system or reclining system or a support and which avoids the disadvantages of the prior art.

The present invention achieves the objects that have been posed by providing a method of determining the location and place of a person's pelvis according to claim 1 and a device for determining the location and place of a person's pelvis having the features of claim 8.

In a further aspect, the invention provides a computer program product according to claim 6, and a computer-readable data carrier on which the computer program product is stored.

The method according to the invention comprises the following steps: detecting sifting pressures/contact pressures exerted on the seat, the lounger or the support by the ischial tuberosities and the coccyx of the person sifting essentially upright or by the iliac crests and the pubic bone of the person lying in a prone position by means of a surface sensor arranged on the seat, the lounger or the support; determining, by a computer unit, a first position on the surface sensor at which a first peak sifting pressure/peak contact pressure exerted by a first ischial tuberosity/iliac crest of the person is reached; determining, by the computer unit, a second position on the surface sensor at which a second peak sifting pressure/peak contact pressure exerted by a second ischial tuberosity/iliac crest of the person is reached, the second position being within a predetermined distance range from the first position; determining, by the computer unit, a distance between the first position and the second position; determining, by the computer unit, a third position on the surface sensor at which a third peak sitting pressure/peak contact pressure exerted by the coccyx/pubic bone of the person is reached, the third position being between the first position and the second position, and the third position being substantially on their common connecting line or within a predetermined distance range on a line running essentially perpendicularly to the connecting line; limiting, by the computer unit, an area of the surface sensor which detects sitting and contact pressures; and evaluating, by the computer unit, the sitting and contact pressures of the person's pelvis in the restricted area detecting sitting and contact pressures.

Due to the method according to the invention, it is possible to determine the location and the position of the person's pelvis on a seat or a lounger at any time, whereby not only individual movements of the person around one of his or her body axes can be detected, but in particular also mixed movements of the initially mentioned kind. For this purpose, the above-mentioned three positions and the magnitude of the sitting and contact pressures exerted by the person's pelvis on the surface sensor are determined. This is preferably performed by the computer unit by way of a pattern recognition of the maxima of the sitting and contact pressures. It is advantageous that, besides, the maxima of the sitting and contact pressures allow conclusions to be drawn about the weight force of the upper body and, depending on the design of the seat with or without armrests, including/excluding the weight force of the person's upper extremities, since, in an essentially upright sitting position, the ischial tuberosities transfer the bulk of the upper body weight to the surface sensor. Smaller weight force proportions, which, in this sitting position, are transferred from the person's lower extremities to the surface sensor and are noticeable in the form of an offset in the sitting and contact pressures, can be compensated for by the computer unit due to the restricted detecting area. If the surface sensor is arranged on a back element of a seat, in particular for backward-leaning positions of the back element, pressure fluctuations caused by the person's respiration can, in addition, be detected dynamically by the surface sensor. In this way, changes in breathing, e.g., tachypnea, can be detected.

Since the sitting and contact pressures of the pelvis, the coccyx and the sacrum are detected exclusively in the area detecting sitting and contact pressures, the advantage is included that pressures exerted on other areas of the surface sensor will not influence the sitting and contact pressures exerted by the person's pelvis on the surface sensor. If the device is used, for example, in hospital beds, objects located on the hospital bed, e.g., bags from visitors, equipment from nurses or doctors, have no bearing on the evaluation of the sitting and contact pressures of the pelvis of the patient located on the hospital bed, as it essentially takes into account the geometry of the pelvis and also remains unaffected by partly fixed malpositions of the spine, which require a support on the backrest. The location and the position of the pelvis can thus be determined precisely for every position of the person on the surface sensor.

It is a further advantage that, by means of the method according to the invention, a relative angle can be determined between the seat element, in particular its seating surface, and the back element, independently of a location-related weight force influence and independently of a support of the person's upper body weight on the back element. This is achieved in that a position can be assigned by the computer unit to each sitting and contact pressure arising in the area of the surface sensor which detects sitting and contact pressures, and a pressure distribution is thus known for every position of the sitting and contact pressure.

In order to be able to narrow down the area of the surface sensor which detects sitting and contact pressures, the computer unit runs an iterative algorithm which depicts the Mandelbrot set using a recursive sequence zₙ₊₁=zₙ²+C, with an initial condition z₀=0. For this purpose, the surface sensor is perceived as a Gaussian plane in which the sitting and contact pressures exerted by the person's pelvis are points C in the Gaussian plane. By inserting different points C into the recursive sequence, it can be ascertained which points C of the Gaussian plane belong to the Mandelbrot set and which do not. In this case, the Mandelbrot set includes those points for which the sequence remains limited, i.e., converges, i.e., increasingly approaches a limit value. For a point C in the Gaussian number plane with the coordinates C(0/0), a circular area detecting seating and contact pressures arises, since the sequence is reduced to zₙ₊₁=zₙ². At points C outside of this circular area, the numbers tend to infinity and the sequence is no longer limited. By mapping the Mandelbrot set by the recursive sequence, it is possible to map the three-dimensional structure of the person's pelvis two-dimensionally on a flat contact surface, for example on the surface sensor. In doing so, the two-dimensional structure of the pelvis essentially corresponds to the shape of a cardioid, which can be represented by the Mandelbrot set. The two-dimensional cardioid shape of the pelvis is created by the rolling process of the latter on the flat contact surface. Starting from a first location of the pelvis, which corresponds to an upright sitting position of the person, the pelvis of this person is tilted forwards around the horizontal axis by 90° into a second location and then backwards into a third location. In order to reach the third location, the pelvis is tilted backwards around the horizontal axis by 90° relative to the first location of the pelvis or, respectively, by 180° relative to the second location of the pelvis. The third location corresponds to a horizontal dorsal position of the person. Starting from the horizontal dorsal position, the pelvis is rolled over the iliac crests on both sides, which corresponds to a mixed rotation of the pelvis or a pure rotation around the longitudinal axis in the reclining position. Thus, a cardioid shape is formed at the connection of all contact points on the flat contact surface. The cardioid thus corresponds to a two-dimensional representation of an outer contour of the person's three-dimensional pelvis. In this case, the sacral structure of the pelvis is depicted also two-dimensionally inside the cardioid.

Depending on the number of iterations, which determines the shape and magnitude of the area detecting sitting and contact pressures and thus the shape and magnitude of the Mandelbrot set, the sitting and contact pressures exerted by the person's pelvis on the surface sensor occur within said cardioid. Few iterations, for example two, result in a larger area detecting sitting and contact pressures, in this case an elliptical area, whereas a large number of iterations produce a more limited, e.g., a cardioid-shaped, area detecting sitting and contact pressures. For positioning the person's pelvis by means and methods known from the prior art, for example those of A 50386/2019, a higher number of iterations is to be preferred over a lower number. With as little as five iterations, a cardioid-shaped limited area detecting sitting and contact pressures can be mapped, which is sufficient for positioning the pelvis by the above-mentioned means and methods. When a predetermined number is reached, the currently running iteration is interrupted. Said number of iterations can be specified and set on the computer unit, for example, by third parties, e.g., by qualified personnel such as nurses or doctors. However, the number of iterations depends primarily on the intended use of the device. If integrated in operating beds, it is necessary to detect the sitting and contact pressures as accurately as possible, which is why a higher number of iterations is chosen for this.

The steps according to the invention mentioned above are implemented by a computer program product which comprises commands which, when the computer unit executes the program, prompt said unit to carry out these steps. In this case, the computer program product is stored on a computer-readable data carrier.

With the support of the program, settings can be made on the device which allow the device to be adapted for its respective intended use, such as, for example, the previously mentioned setting via the number of iterations of the algorithm.

The device for determining the place and location of a pelvis of a person sitting on a seat or sitting or lying on a lounger or a support comprises a computer unit and a surface sensor connected to the computer unit. The surface sensor is arranged on the seat, the lounger or the support and is designed for detecting sitting and contact pressures which are exerted on the surface sensor by the pelvis, coccyx and sacrum of the person, with the computer unit being designed for carrying out the steps of the above-mentioned method according to the invention.

It is advantageous that the device can be integrated into existing seats or loungers or can be used as a seat cover on a chair or bed. This results in a variety of possible applications, wherein the device is not limited to the following examples. To give examples, the device could be integrated into the following entities: into office chairs, wheelchairs, vehicle seats, child restraint systems, workout equipment, treatment beds, mattresses, and/or it could be used in the rehabilitation and therapy sector, in particular in standing beds, standing boards and/or operating beds. The integration of the present invention into operating beds makes sense particularly for procedures carried out in a prone or dorsal position, as they are often performed with inclined operating beds, as a result of which an undesirable relocation of the person located on the operating bed may happen, or, respectively, consequences of an incorrect positioning of the pelvis in relation to the neutral orientation of the spine, in which the spine, especially in the lumbar region, is forced into a compressed or stretched position. Surgery on a pelvis positioned in this way can lead to a variety of problems. By means of the present invention, a displacement or rotation of the person's pelvis can be identified quickly by detecting displacements in particular of the maximum sitting and contact pressures on the surface sensor. Displacements or, respectively, rotations of the pelvis can be counteracted and the original position of the pelvis can be restored by means and methods known from the prior art, e.g., by the device cited at the beginning. Thus, in the course of an operation, a procedure can be carried out with the best possible precision, and possible surgical risks and, respectively, painful consequences of an incorrect positioning of the functional spine can be minimized or even eliminated in particular by supporting the lumbar spine in a way that is matched to the position of the pelvis.

In a preferred embodiment of the invention, the surface sensor is an array of sensors selected from the group consisting of mechanical, electrical, pneumatic or hydraulic sensors. As a result, the position of the pelvis can be detected in every position of the person on the seat or lounger. By varying the number of sensors, the accuracy of the detection of sitting and contact pressures by the sensors can be influenced. A large number of sensors enable precise detection of the sitting and contact pressures of the person's pelvis.

In a preferred embodiment of the invention, the surface sensor is shaped in such a way that it adapts at least partially to the person's pelvis. Sitting and contact pressures can thus be detected with higher sensitivity, whereby even slight shifts in the centre of gravity, particularly in the area of the pelvic body section, can be detected by the sensors.

The invention is now explained in further detail with reference to the drawings using non-limiting exemplary embodiments.

FIG. 1 is a block diagram which illustrates steps of a method according to claim 1 of the invention in a preferred order.

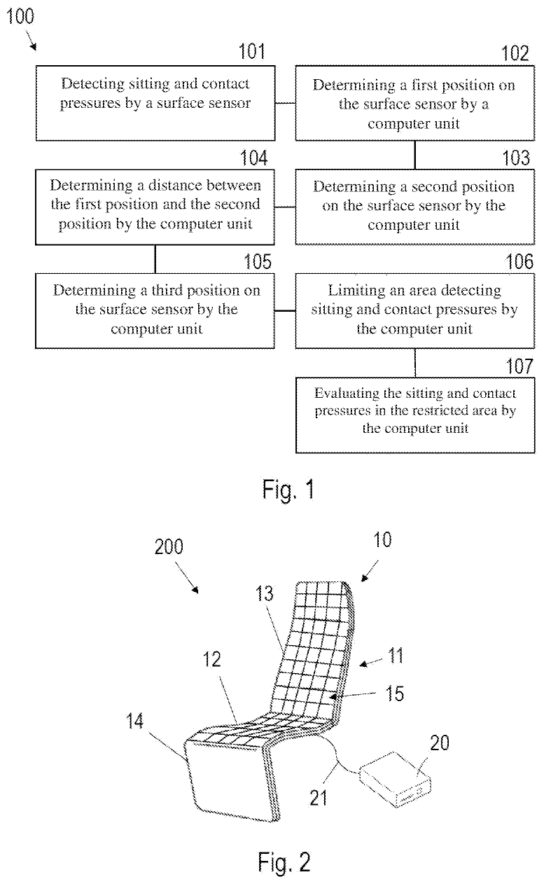

FIG. 2 shows a schematic perspective illustration of a seat with an integrated surface sensor according to claim 8 of the invention.

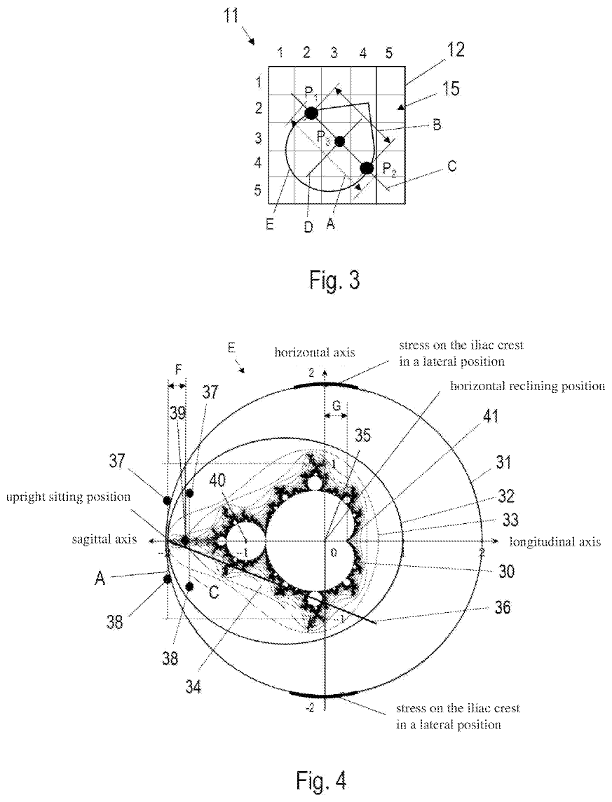

FIG. 3 shows a schematic illustration of a seating surface of the seat depicted in FIG. 1 in which a first position, a second position and a third position according to claim 1 of the invention are illustrated.

FIG. 4 shows an area of the surface sensor which detects sitting and contact pressures.

Hereinafter, reference is made to FIGS. 1 to 3. Referring initially to FIG. 2, a device 200 according to the invention for determining the place and location of a pelvis of a person sitting on a seat 10 or sitting or lying on a lounger or a support is shown. The lounger, the support and the person are not shown in the figures. The device 200 comprises a computer unit 20 and a surface sensor 11 connected to the computer unit 20. The surface sensor 11 is arranged on a seat element 12 and in a back element 13 of the seat 10, in particular integrated therein, wherein a leg element 14 of the seat 10 has no surface sensor 11. In a further embodiment, which is not shown, the leg element 14 can also have a surface sensor 11, in addition to the seat element 12 and the back element 13. In still further embodiments, which are also not shown, only the seat element 12 or the back element 13 can have a surface sensor 11. The above-mentioned embodiments are combinable, meaning that, for example, the leg element 14 and the seat element 12 have a surface sensor 11 and the back element 13 has no surface sensor 11. The surface sensor 11 exemplified in FIG. 2 has comparatively large-scale individual sensors, for which reason the determination of distances can be done only relatively roughly. Therefore, the surface sensors used in connection with the invention can also have far more individual sensors in a finer grid or array than what is shown in FIG. 2.

In order to achieve high sensitivity of the sensors 15, the surface sensor 11 is shaped in such a way that it adapts at least partially to the person's pelvis. As a result, the surface sensor 11, which is designed for detecting sitting and contact pressures exerted on the surface sensor 11 by the pelvis, coccyx and sacrum of the person, can detect the sitting and contact pressures with a high level of precision. As can be seen in FIG. 2, the surface sensor 11 is an array of sensors 15 selected from the group consisting of mechanical, electrical, pneumatic or hydraulic sensors. In particular, the surface sensor is a two-dimensional array of sensors 15 which completely occupies the seat element 12 and the back element 14 of the seat 10. The number of sensors 15 arranged in the array can vary and is not limited to a specific number. An array with 5×5 sensors 15 is illustrated in FIG. 3. As a result, the position of the sitting and contact pressures exerted by the pelvis, coccyx and sacrum of the person, and thus every position of the person's pelvis, can be determined with sufficient accuracy. The sensors 15 of the surface sensor 11 are preferably designed as flat chambers filled with a fluid, e.g., air or water. In this case, the surface sensor 11 is connected to the computer unit 20 via fluid-carrying channels, which are not shown in the figures. Electrical or mechanical sensors can also be used for detecting the sitting and contact pressures and are therefore not limited to the sensors mentioned above by way of example. Thus, the sensors 15 can also be strain gauges which can be arranged in an array. In this case, the computer unit 20 is electrically connected to the surface sensor 11.

Hereinafter, reference is made to FIGS. 1 and 3. In FIG. 1, a block diagram is shown which illustrates steps 101, 102, 103, 104, 105, 106, 107 of a method 100 according to the invention for determining the place and location of a pelvis of a person sitting on a seat 10 or sitting or lying on a lounger or a support. In a first step 101 of the method 100, sitting pressures/contact pressures exerted on the seat 10, the lounger or the support by the ischial tuberosities and the coccyx of the person sitting essentially upright or by the iliac crests and the pubic bone of the person lying in a prone position are detected by means of a surface sensor 11 arranged on the seat 10, the lounger or the support. In a next step 102, a first position P₁ on the surface sensor 11 at which a first peak sitting pressure/peak contact pressure exerted by a first ischial tuberosity/iliac crest of the person is reached is determined by the computer unit 20. In FIG. 3, the first position P₁ is detected by the sensor in row 2 and column 2 of the array by way of example. In a further step 103, a second position P₂ on the surface sensor 11 at which a second peak sitting pressure/peak contact pressure exerted by a second ischial tuberosity/iliac crest of the person is reached is determined by the computer unit 20, the second position P₂ being within a predetermined distance range A from the first position P₁. In FIG. 3, the second position P₂ is detected by the sensor in row 4 and column 4 of the array. The predetermined distance range A is defined by the geometric arrangement of the ischial tuberosities or, respectively, the iliac crests of the person and is 100 mm to 150 mm for the ischial tuberosities and 200 mm to 250 mm for the iliac crests in the prone position. The specified distance range A is considered as exemplary, since, in addition to gender-specific differences, other parameters such as age or pathological changes, especially in the area of the coccyx of a person, also have an influence on the geometry of the pelvis or, respectively, the sacral structure, and thus on the distance range A. In a subsequent step 104 of the method 100, a distance B between the first position P₁ and the second position P₂ is determined by the computer unit 20. In this case, this distance B corresponds to a true distance between the first ischial tuberosity/iliac crest and the second ischial tuberosity/iliac crest of the person, the distance B between the iliac crests being greater than between the ischial tuberosities. The term “true distance” is understood to refer to the actual distance between the ischial tuberosities or, respectively, the iliac crests which corresponds to the respective pelvis of the person and is detected by the surface sensor 11.

If the first position P₁, the second position P₂ and the distance B between said positions P₁, P₂ have been determined by the computer unit 20, a third position P₃ on the surface sensor 11 at which a third peak sitting pressure/peak contact pressure exerted by the coccyx/pubic bone of the person is reached is subsequently determined by the computer unit 20. In this case, the third position P₃ is between the first position P₁ and the second position P₂, the third position P₃ being substantially on their common connecting line C or within a predetermined distance range, not shown, on a line D running essentially perpendicularly to the connecting line C. In FIG. 3, the third position P₃ is detected by the sensor in row 3 and column 3 of the array.

After the third position P₃ has been determined, a restricted area E detecting sitting and contact pressures is determined by the computer unit 20 in a next step 106, with this area being shown schematically in FIG. 3 and in the form of a Mandelbrot set 30 in FIG. 4. In order to limit the area E detecting sitting and contact pressures, the computer unit 20 executes an iterative algorithm which maps said Mandelbrot set 30.

In a final step 107 of the method 100, the sitting and contact pressures of the person's pelvis in the restricted area E detecting sitting and contact pressures are evaluated by the computer unit 20. Sitting and contact pressures outside of this range E are not evaluated any further by the computer unit 20.

In this case, the computer unit 20 of the device 100 is designed for carrying out the steps 101, 102, 103, 104, 105, 106, 107 of the above-mentioned method 200, with the order of the method 100 not being limited to the preferred order as shown in FIG. 1. For example, the steps 102, 103 of the method 100 can also take place in reverse order.

Hereinafter, reference is made to FIG. 4. FIG. 4 shows the restricted area E detecting sitting and contact pressures or, respectively, the Mandelbrot set 30 in different shapes and sizes.

Furthermore, FIG. 4 shows the sitting and contact pressures 37, 38, 39 that occur in the restricted area E detecting sitting and contact pressures and are exerted on the surface sensor 11 by the pelvis and the coccyx or sacrum, based on which the place and, in addition, also the location of the pelvis can be determined. As already mentioned, the area E detecting sitting and contact pressures is limited by the above-mentioned iterative algorithm. For the sake of illustration, a first iteration 31 of the algorithm which forms a circular area E detecting sitting and contact pressures, a second iteration 32 which forms an elliptical area E detecting sitting and contact pressures, and a third iteration 33 should be mentioned. As can be seen in FIG. 4, the area E detecting sitting and contact pressures is increasingly restricted with increasing iterations, with the area E detecting sitting and contact pressures having essentially the shape of a cardioid or, respectively, the shape of the Mandelbrot set 30 already at five iterations 34. According to the rectangular coordinate system of the pelvic structure, the sagittal axis in the range from −2 to 0 and the longitudinal axis in the range from 0 to 2 are plotted on the abscissa, and the horizontal axis of the person's pelvis is plotted on the ordinate. An origin 35, which is illustrated at a crossing point of the abscissa and the ordinate, represents a stable equilibrium position with stress on the sacral structure and is shown at a distance G from an edge 41 of the Mandelbrot set 30. The origin 35 corresponds to the centre of gravity of the weight of the person's body section and, when the surface sensor 11 is integrated, represents a horizontal dorsal position of the person or, respectively, that point is loaded with maximum contact pressure by the sacrum, provided that the longitudinal axis or, respectively, the frontal plane of the person's upper body has a parallel orientation relative to a support structure, such as, for example, a standing board not shown in the figures.

During a rolling process of the pelvis, i.e., a rotation of the pelvis around the horizontal axis, the ischial tuberosities first exert peak sitting pressures 37, 38 on the surface sensor 11, starting from an upright sitting position of the person on the surface sensor 11. In doing so, the peak sitting pressures 37, 38 are at the above-mentioned distance A from one another and arise outside of the restricted area E detecting sitting and contact pressures. If asymmetric stress is put on the ischial tuberosities, pressure differences between the peak sitting pressures 37, 38 can be detected by the surface sensor 11. Due to these pressure differences, an angle of rotation of the pelvis around the sagittal axis of the person can be determined by the computer unit 20, whereby the true length of the ischial tuberosities, in case of a prone position the true length of the iliac crests, and from this a radius of the circular area E of the first iteration 31 of the algorithm which detects sitting and contact pressures can be determined by the computer unit 20. In case of a tilt of the pelvis around the horizontal axis of essentially 10° and more, depending on the flexibility of the substructure and any pathological changes in the coccyx, the coccyx of the person exerts a sitting pressure 39 on the surface sensor 11, which is located essentially on the common connecting line C formed by the peak sitting pressures 37, 38. In this case, the sitting pressure 39 can be at a distance F from the peak sitting pressures 37, 38. In this location of the pelvis, the distance range A of the peak sitting pressures 37, 38 corresponds to half the radius of the circular area E of the first iteration 31 of the algorithm, which area detects sitting and contact pressures.

The sitting pressure exerted on the surface sensor 11 by the coccyx increases during the rolling process up to a transition 40 at which the sacrum of the person exerts a contact pressure on the surface sensor 11. In the horizontal reclining position of the person, i.e., a location of the pelvis tilted around the horizontal axis by 90°, the contact pressure exerted by the sacrum reaches its maximum at the origin 35. Starting from the horizontal reclining position of the person, if said person moves to the side, i.e., laterally rolls his or her pelvis in a mixed movement or a rotation around the longitudinal axis so that a lateral position is adopted, the pelvis exerts contact pressures on the surface sensor 11. These contact pressures are illustrated in FIG. 4 by a line 36 showing a mixed sideways movement of the person by 30°. If a person in a horizontal reclining position rotates by 180° in a mixed movement, the area detecting sitting and contact pressures would only detect sitting and contact pressures arising within the first 90° of that rotation, i.e., within the circular area E of the first iteration 31 which detects sitting and contact pressures. Sitting and contact pressures arising on the surface sensor 11 and going beyond this 90° rotation and thus beyond the circular area E of the first iteration 31 which detects sitting and contact pressures would not be detected by said area. To still detect such movements of the person, the computer unit 20 is designed for storing the area E detecting sitting and contact pressures. If sitting and contact pressures exerted by the pelvis arise on a peripheral region of the circular area E of the first iteration 31 which detects sitting and contact pressures, a second area detecting sitting and contact pressures, which is not shown in the figures, is depicted adjacent to the area E detecting sitting and contact pressures. In this case, the second area detecting sitting and contact pressures is a copy of the area E which detects sitting and contact pressures. This process can be repeated several times by the computer unit so that movements or, respectively, sitting and contact pressures exerted by the pelvis outside of the area E detecting sitting and contact pressures can be detected for every movement of the person.

With further lateral rolling of the pelvis in a movement around the longitudinal axis or a mixed movement, i.e., the person adopting a horizontal prone position, only the pelvic structure is stressed. The pubic bone and the iliac crests can exert maximum contact pressures on the surface sensor 11. In mathematical terms, these contact pressures correspond to the contact pressures of the person in a dorsal position, wherein the sacral structure is stressed in the dorsal position and the pelvic structure is stressed in the prone position. With a maximum load on the sacrum in a horizontal dorsal position at the origin 35 or, respectively, on the pubic bone in a horizontal prone position in an area of the transition 40, the stable equilibrium position is reached.

The distances F and G are essentially the same size, although they can differ, for example, due to a pathological change in the anatomy of the person in the area of the coccyx. If the distances F and G are unequal and other characteristic points or, respectively, pressure areas not shown in the figures are known, the distance F can be corrected by calculation and exact conclusions can still be drawn about an ergonomically correct or incorrect sitting or reclining position.

It may be mentioned that the method according to the invention provides mathematically exact results also when the sensitivity of the sensors is reduced to a very narrow range, for example for cost reasons, and consequently only no or maximum pressure values are detected by the sensor.

Furthermore, it may be mentioned that the surface sensor can also be integrated into the seating surface only directly in the contact area of the ischial tuberosities and the coccyx and sacrum. This is possible because, for the calculation method, based on an evaluable parameter, only geometric fixed points dependent therefrom are used for assessing the location of the pelvis, but they do not actually have to be physically present. This would be interesting, for example, for determining a location for pure seating systems.

1.-10. (canceled)

11. A method of determining the place and location of a pelvis of a person sitting on a seat or sitting or lying on a lounger or a support, wherein the method comprises the following operations:
a) detecting sitting pressures/contact pressures exerted on the seat, the lounger or the support by the ischial tuberosities and the coccyx of the person sitting essentially upright or by the iliac crests and the pubic bone of the person lying in a prone position by a surface sensor arranged on the seat, the lounger or the support;
b) determining, by a computer unit, a first position (P₁) on the surface sensor at which a first peak sitting pressure/peak contact pressure exerted by a first ischial tuberosity/iliac crest of the person is reached;
c) determining, by the computer unit, a second position (P₂) on the surface sensor at which a second peak sitting pressure/peak contact pressure exerted by a second ischial tuberosity/iliac crest of the person is reached, the second position (P₂) being within a predetermined distance range A from the first position (P₁);
d) determining, by the computer unit, a distance B between the first position (P₁) and the second position (P₂);
e) determining, by the computer unit, a third position (P₃) on the surface sensor at which a third peak sitting pressure/peak contact pressure exerted by the coccyx/pubic bone of the person is reached, the third position (P₃) being between the first position (P₁) and the second position (P₂), and the third position (P₃) being substantially on their common connecting line (C) or within a predetermined distance range on a line (D) running essentially perpendicularly to the connecting line (C);
f) limiting, by the computer unit, an area (E) of the surface sensor which detects sitting and contact pressures; and
g) evaluating, by the computer unit, the sitting and contact pressures of the pelvis in the restricted area (E) detecting sitting and contact pressures.

12. A method according to claim 11, wherein, for narrowing down the area (E) detecting sitting and contact pressures, the computer unit runs an iterative algorithm which depicts the Mandelbrot set.

13. A method according to claim 12, wherein the iteration is interrupted when a predetermined number is reached.

14. A method according to claim 13, wherein the shape and the magnitude of the restricted area (E) of the surface sensor which detects sitting and contact pressures depend on the number of iterations.

15. A method according to claim 11, wherein, for determining the first position (P₁), the second position (P₂) and the third position (P₃), a pattern recognition of the maxima of the sitting and contact pressures is performed by the computer unit.

16. A non-transitory storage medium having stored therein instructions that are executable by one or more hardware processors to perform operations comprising prompting the computer unit to carry out the operations recited in claim 11.

17. A device for determining the place and location of a pelvis of a person sitting on a seat or sitting or lying on a lounger or a support, comprising a computer unit and a surface sensor connected to the computer unit and arranged on the seat, the lounger or the support and being operable to detect sitting and contact pressures which are exerted on the surface sensor by the pelvis, with the computer unit operable to carry out the operations of the method according to claim 11.

18. A device according to claim 17, wherein the surface sensor is an array of sensors selected from the group consisting of mechanical, electrical, pneumatic or hydraulic sensors.

19. A device according to claim 17, wherein the surface sensor is shaped in such a way that it adapts at least partially to the pelvis.
